(19) United States
(12) Patent Application Publication
(10) Pub. No.: US 2026/0367059 A1
(43) Pub. Date: Nov. 19, 2026

(54) ELECTROMAGNETIC DETECTION OF A STATE OF AN EGG

(71) Applicants: Zen Genetics Ltd., Tel-Aviv (IL); Technion Research & Development Foundation Limited, Haifa (IL)

(72) Inventors: Dor GREEN, Tel-Aviv (IL); Israel SCHECHTER, Haifa (IL)

(73) Assignees: Zen Genetics Ltd., Tel-Aviv (IL); Technion Research & Development Foundation Limited, Haifa (IL)

(21) Appl. No.: 19/148,017

(22) PCT Filed: Jan. 14, 2024
(86) PCT No.: PCT/IL2024/050050

§ 371 Date: Jul. 15, 2025

Related U.S. Application Data
(60) Provisional application No. 63/439,123, filed on Jan. 15, 2023.

(51) Int. Cl. G01N 33/08; G01N 21/31; G01N 21/47; G01N 21/64; G01N 21/65

(52) U.S. Cl. CPC G01N 33/08; G01N 21/31; G01N 21/474; G01N 21/6486; G01N 21/65; G01N 2201/064

(57) ABSTRACT

A method and system for detecting a state of an egg. Scan values are obtained for multiple scan locations on a shell of an egg, irradiation angles, and/or collection angles, at each the egg is irradiated and reflectance and/or emission is captured at a collection point and measured by a time-gated detector for one or more arrival times relative to an irradiation time. The scan values are analyzed to determine information of the egg's germinal disc and a state of the egg is determined according to the information.

### RELATED APPLICATION/S

This application claims the benefit of priority of U.S. Provisional Patent Application No. 63/439,123 filed on Jan. 15, 2023, the contents of which are incorporated herein by reference in their entirety.

### FIELD AND BACKGROUND OF THE INVENTION

Some embodiments described in the present disclosure relate to electromagnetic detection and, more specifically, but not exclusively, to determination of a state of an egg using electromagnetic detection.

Each year hundreds of billions of eggs are produced in the world. Duration of incubation for chicken eggs hatching takes about 21 days and consumes time and energy. A significant percentage, typically 10 to 40%, of eggs is infertile. These useless eggs consume space and energy within an incubator and can also cause contamination of other eggs. In such an industry, efficient quality control and limiting production costs are required.

### SUMMARY OF THE INVENTION

It is an object of the present disclosure to describe a system and a method for electromagnetic detection of a state of an egg.

The foregoing and other objects are achieved by the features of the independent claims. Further implementation forms are apparent from the dependent claims, the description and the figures.

According to one aspect of some embodiments of the disclosed subject matter there is provided a method for detecting a state of an egg, comprising: obtaining a plurality of scan values by performing, over a plurality of iterations respective of at least one of a plurality of scan locations on a shell of an egg, a plurality of irradiation angles, and a plurality of collection angles: irradiating the egg with electromagnetic radiation at a respective location of the plurality of scan locations and a respective one of the plurality of irradiation angles; capturing and measuring intensity of electromagnetic radiation emanating from the egg at a collection point corresponding to the respective location of irradiance and a respective one of the plurality of collection angles, using a time-gated detector; and calculating and storing a respective scan value for the respective location and angles indicative of the intensity measured for at least a portion of a plurality of arrival times of the captured electromagnetic radiation to the time-gated detector relative to a time of irradiation; analyzing the plurality of scan values for at least the portion of the plurality of arrival times to determine information of a germinal disc of the egg; and determining a state of the egg according to the information.

Optionally, for at least one portion of the plurality of scan locations, a respective portion of the plurality of scan values are analyzed to determine a local extremum indicative of incidence on a line intersecting with the germinal disc, wherein at least another portion of the plurality of scan locations are determined in accordance with a respective one of the at least one portion of the plurality of scan locations at which the local extremum is obtained.

Optionally, the plurality of scan locations on the shell of the egg being positioned along one of a longitudinal axis and a latitudinal axis of the egg and spaced apart from one another by a distance ranging between about 0.5 millimeter and about 1 millimeter.

Optionally, the irradiating comprising applying a plurality of electromagnetic pulses having a width of between about 100 femtoseconds and about 100 picoseconds.

Optionally, the irradiating comprising applying at least one wavelength at which a difference between at least one of a reflectance and emission of electromagnetic radiation from the germinal disc, and at least one of the reflectance and emission from a matrix of the germinal disc within the egg, is discernable.

More optionally, the irradiating comprising applying a plurality of wavelengths, wherein for at least a portion of the plurality of wavelengths the difference is by a first magnitude, wherein for at least an additional portion of the plurality of wavelengths the difference is by a second magnitude distinct from the first magnitude.

More optionally, the portion comprising a wavelength of about 490 nanometers, wherein the additional portion comprising a wavelength of about 540 nanometers.

More optionally, the method further comprising normalizing captured intensities for the portion using respective captured intensities for the additional portion.

Optionally, the collection point being removed from the respective location of irradiance by a distance of between about 0.5 millimeters and about 5 millimeters.

Optionally, the irradiating and capturing further comprising preventing at least in part from reflectance of electromagnetic radiation from the shell of the egg to reach the collection point.

Optionally, analyzing the plurality of scan values and determining the state of the egg comprising: determining a peak and a width thereof along at least one line trajectory on the shell of the egg; and determining a fertility state of the egg according to at least one of: determining a diameter of the germinal disc of the egg according to a maximal width determined from the at least one line trajectory, and determining a lower bound of the diameter in response to the width along a respective one of the at least one line trajectory exceeding a threshold.

Optionally, obtaining the plurality of scan values is performed at a spatial resolution for which a morphology of the germinal disc is discernable, wherein analyzing the plurality of scan values comprising detecting at least one morphological characteristic of the germinal disc, wherein determining the state of the egg comprising determining a health condition of the egg.

Optionally, the egg is a freshly laid egg for which a time elapsed from laying is less than an hour.

Optionally, the plurality of iterations being performed respective of at least one of the plurality of scan locations and the plurality of collection angles, and wherein the irradiating being at an irradiation angle of about 0°.

Optionally, the plurality of iterations being performed respective of at least one of the plurality of scan locations and the plurality of irradiation angles, and wherein the capturing being at a collection angle of about 30°.

According to another aspect of some embodiments of the disclosed subject matter there is provided a system for detecting a state of an egg, comprising: an irradiation source adapted for irradiating an egg with electromagnetic radiation at a respective location of a plurality of scan locations on a shell of an egg and a respective one of the plurality of irradiation angles; a time-gated detector adapted for capturing and measuring intensity of electromagnetic radiation emanating from the egg at a collection point corresponding to the respective location of irradiance and a respective one of the plurality of collection angles; a processing circuitry coupled to a memory and adapted to execute a code for: obtaining a plurality of scan values by operating the irradiation source and time-gated detector over a plurality of iterations respective of at least one of the plurality of scan locations, the plurality of irradiation angles, and the plurality of collection angles; analyzing the plurality of scan values for at least the portion of the plurality of arrival times to determine information of a germinal disc of the egg; and determining a state of the egg according to the information.

Optionally, the system further comprising a blocking shield for preventing from reflectance of electromagnetic radiation from the shell of the egg to reach the collection point.

Optionally, the system further comprising an objective lens for collecting and transferring the electromagnetic radiation emanating from the egg at the collection point to the time-gated detector.

Optionally, the system further comprising a translation stage for positioning the egg relative to the irradiation source for the irradiating at the plurality of scan locations.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

Unless otherwise defined, all technical and/or scientific terms used herein have the same meaning as commonly understood by one of ordinary skill in the art to which embodiments. Although methods and materials similar or equivalent to those described herein can be used in the practice or testing of embodiments, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments are herein described, by way of example only, with reference to the accompanying drawings. With specific reference now to the drawings in detail, it is stressed that the particulars shown are by way of example and for purposes of illustrative discussion of embodiments. In this regard, the description taken with the drawings makes apparent to those skilled in the art how embodiments may be practiced.

In the drawings:

FIG. 1 is a schematic illustration of an exemplary measurement arrangement for electromagnetic detection of a state of an egg, according to some embodiments;

FIG. 2 is a schematic graph representing exemplary scan profiles at a latitudinal scan line of a fertile and a non-fertile egg, according to some embodiments;

FIG. 3 is a flowchart schematically representing an optional flow of operations for electromagnetic detection of a state of an egg, according to some embodiments; and FIG. 4 is a schematic block diagram of an exemplary system for electromagnetic detection of a state of an egg, according to some embodiments.

### DESCRIPTION OF SPECIFIC EMBODIMENTS OF THE DISCLOSURE

Some embodiments described in the present disclosure relate to electromagnetic detection and, more specifically, but not exclusively, to determination of a state of an egg using electromagnetic detection.

The germinal disc, also referred to as blastodisc, is a flattened, disc-like region of cells from which an embryo develops in a fertilized egg of various species, such as birds and the like. Typically, the germinal disc sits on the surface of the yolk of the egg and appears as a small white circular spot on top of the yellow yolk matrix.

The germinal disc of fertile eggs is circa 2-3 times larger than that of non-fertile eggs. In addition, there are other morphological differences in the germinal disc of fertile and non-fertile eggs, which are related to fertility and to other conditions of the egg, such as health and/or the like, for example, irregularities in shape and/or boundary line of the germinal disk, such as protrusions and/or spikes and/or likewise deformations may be caused by a number of diseases and thus indicate their presence.

While inspection of the germinal disc provides information on fertility and/or health condition of the egg at hand, which is useful for conservation of resources in egg production and/or prevention of diseases and/or contaminations from spreading, there are also however several shortcomings and difficulties involved, in particular, the fact that a high share of eggs do not survive the trauma induced due to the penetration of the shell of the egg entailed in the process, even where only a microscopical portion of the shell is removed for the purpose of conducting the inspection and reattached thereafter.

In view of the foregoing, it is therefore desired that determination of a fertility state and/or health condition of an egg, e.g., based on detection of a size of the germinal disc and/or its morphology, may be performed in a non-invasive manner, so as to avoid attrition of viable eggs due to their examination while ensuring quality control and efficiency.

In accordance with some embodiments of the disclosed subject matter, inter-egg content information may be obtained using electromagnetic imaging, where a target egg is irradiated by one or more beams of electromagnetic radiation which pass through the egg's organelles and arrive at the germinal disk within the egg to be detected. The irradiated beams may be reflected by, and/or absorbed by, and/or transmitted through at least one of the organelles and captured by a detector suitable for measuring intensity of electromagnetic radiation emanating from the egg and/or its shell's surface. Additionally or alternatively, emission of electromagnetic radiation from at least one of the organelles due to irradiation of the egg, such as fluorescence and/or Raman scattering, may similarly travel towards and be captured at and measured by the detector. In such manner, a plurality of measurements of reflectance and/or emission of electromagnetic radiation at a plurality of locations on a shell of an egg, a plurality of collection angles, and/or a plurality of irradiation angles may be performed. Patterns and/or differences in the intensities measured as the location, angle of collection, and/or angle of irradiation vary may be analyzed and accordingly a determination regarding a fertility state and/or health condition of the egg may be made.

As used herein, the term irradiation point refers to a point on the shell of the egg where the irradiance beam from the source of electromagnetic radiation, e.g., a laser and/or a likewise light source that is utilized for reflectance inducement and measuring, hits the eggshell's surface. Throughout the present disclosure, the term electromagnetic radiation and the term light may be used interchangeably.

The term collection point as used herein refers to a point on the shell of the egg where the reflectance and/or emission is collected.

The term collection angle as used herein refers to the angle between the following two lines: (a) the line between the collection point and the detector used for measurement; and, (b) the normal of the eggshell's surface at the collection point.

The term irradiation angle as used herein refers to the angle between the following two lines: (a) the line of propagation of the irradiance beam; and, (b) the normal of the eggshell's surface at the irradiation point.

In some embodiments, the capturing of reflected and/or emitted electromagnetic radiation at a respective location of a collection point and/or at a respective collection angle may be performed using a time-gated and/or time resolved detector, such as for example a streak camera and/or the like.

Time domain gated detection as referred to herein is a technique in which the detector can be exposed to the measured signal for a brief interval correlated with a specific region of interest. By controlling a time and duration during which the detector is activated, other interfering signals may be suppressed and/or eliminated and the signal to noise ratio may be improved. For example, time gating and/or likewise time resolved approaches may make use of temporal differences of fluorescence emission and Raman scattering for excluding most of the fluorescence interference from the measurement, using fast detectors to collect information during moments of time where most of the Raman scattering is located.

In some embodiments, an intensity level of captured reflectance and/or emission may be determined for each of one or more arrival times at the time-gated detector relative to the time of irradiation. The time difference and/or delay between the irradiance and capturing may correspond to the propagation of electromagnetic radiation in a target inter-egg media, e.g. the germinal disc. Additionally or alternatively, the temporal resolution of the time-gated detector may correspond to electromagnetic radiation traveling from the irradiation source to penetrate a target egg and then reaching a predetermined inter-egg location, and then reflected from (and/or absorbed in, and/or passing through) said location towards the detector at an interval of less than a spatial resolution resulting from the propagation of electromagnetic radiation in the desired inter-egg media.

In the context of the present disclosure, the term spatial resolution refers to ability to obtain information needed to assess at least one of a fertility and/or other conditions of the egg such as health and/or the like with a sufficiently high signal to noise ratio.

In some embodiments, the irradiating may be by a plurality of short time resolved pulses. The plurality of electromagnetic pulses may have a width ranging between about 100 femtoseconds and about 100 picoseconds, and preferably of about 1 picosecond. The width of the pulses may be determined as a function of the irradiance source being used, as a skilled artisan would readily appreciate. Additionally or alternatively, the irradiating may be by a continuous radiation.

In some embodiments, the irradiating may be by pulses and/or continuous radiation at a wavelength for which the reflectance and/or emission of the germinal disc is discernible from that of its matrix within the egg, e.g., the egg yolk and/or the like. The difference in reflectance and/or emission between the germinal disc and the yolk, which may vary as a function of a particular irradiation wavelength used, is also referred to herein as contrast. The wavelength of irradiance may be of about 490 nanometers, for which the contrast is of a magnitude sufficiently high. For example, a high contrast magnitude may be defined as one which may be obtained where a predetermined threshold being exceeded by a ratio between the reflectance and/or emission arriving from the germinal disc and the reflectance and/or emission arriving from the egg yolk matrix in response to irradiating of the eggshell. The threshold may be, for example, about 1.5 and/or higher.

Additionally or alternatively, multiple wavelengths may be used, simultaneously and/or successively over several measurements, and the various results obtained at each wavelength may be integrated for improving accuracy and/or reliability. For example, the irradiating may be by two wavelengths, one for which the contrast is of a first magnitude, and another for which the contrast is of a second magnitude distinct from the first magnitude, e.g., one of the two wavelengths may be of about 490 nanometers, for which the contrast may be high, and the other one of the two wavelengths may be of about 540 nanometers, for which the contrast may be low. For example, a low contrast magnitude may be defined as one which may be obtained where a ratio between the reflectance and/or emission arriving from the germinal disc and the reflectance and/or emission arriving from the egg yolk matrix in response to irradiating of the eggshell falls short of a predetermined threshold. The threshold may be, for example, about 1.5 and/or lower. The measured intensities at one wavelength may be used in normalization of the measured intensities at another wavelength, such as for example by calculation of a ration therebetween and/or the like, e.g., the measurements at a wavelength of about 490 nanometers may be divided by respective measurements (i.e., with all other parameters being identical) at a wavelength of about 540 nanometers. The ration may be calculated and recorded at each measurement point and/or angle of collection and/or irradiance. Such normalization may compensate for various optical effects that may degrade signal quality.

In some embodiments, the reflectance and/or emission of electromagnetic radiation from the egg may be collected at a distance from the irradiation point. The distance by which the collection point is removed from the irradiation point may range between about 0.5 millimeters and about 5 millimeters, and preferably of about 3 millimeters apart. By spacing the irradiation and collection points apart, saturation of the detector and/or noise such as unwanted scattering and/or the like may be avoided.

In some embodiments, reflectance from the surface of the eggshell may be prevented from reaching the collection point and/or the detector by using a blocking shield, such as for example, a black painted sheet having a same and/or similar curvature as of the shell of the egg in the whereabouts of the measurement location, and/or a likewise light absorbing medium. The blocking shield may be placed between the irradiation and collection points and moved along as the measurement location changes to its corresponding position. The blocking shield may be made for example of paper, plastic, metal, and/or any other non-transparent material. Optionally, the blocking shield may comprise a set of overlapping sheets such that when brought in contact with the egg they take its curved surface shape, so as to avoid a need of fitting the shape of the shield to each individual egg examined. Additionally or alternatively, the gap between the irradiation and collection points may be painted black, in conjugation with the placing of an iris (or two) near the collection point and in front of the detector, where the paint prevents light propagation along the surface, while the iris reduces the light from the irradiation point traveling in air towards the detector. Additionally or alternatively, enhanced temporal resolution of the radiation source and/or detector (i.e. shorter pulses and/or shorter detection gating) may reduce and/or eliminate a need of such mechanical separation, e.g. by separation and suppression of the signal from the irradiation point reaching the detector earlier than the signal from the germinal disc. Additionally or alternatively, the collecting may be performed selectively by optical separation, such as for example using light polarization and/or the like.

In some embodiments, the emanating electromagnetic radiation at a collection point where a measurement being performed may be collected and transferred to the time-gated detector using an objective lens and/or a likewise optical arrangement. The objective lens may be located behind the blocking shield to prevent collection of reflectance from the shell of the egg.

In some embodiments, the plurality of measurements may be two-phased, where a first portion of the measurements is aimed at searching for the germinal disc and a second portion is aimed at determining the size and/or additional morphological information of the germinal disc, once it is located, by detailed scanning. At each stage, the measurements may be performed iteratively for a plurality of locations (i.e., irradiation points), a plurality of collection angles, a plurality of irradiation angles, a plurality of wavelengths, a plurality of pulses, a plurality of time delays and/or arrival times at the detector after irradiation, and/or any likewise adjustable parameters.

The measured value of intensity captured at the detector for a respective location, collection angle, irradiation angle, wavelength, and/or time of arrival may be recorded and a new measurement at a subsequent location, angle, wavelength, and/or time may take place. Additionally or alternatively, a ratio between the captured and irradiated intensities may be calculated and stored for further analysis, once all or a sufficient number of measurements have been completed. Additionally or alternatively, a ration of the measured intensities at different irradiation wavelengths, where applicable, may be calculated and stored for similar purposes.

In some embodiments, at a working environment for detection, an electromagnetic radiation source, a detector, and optionally an objective lens and/or prism may be deployed around and directed towards a viewing field where a levelled and/or planar surface may be provided. Optionally the surface may be comprised and/or coupled to an XYZ stage and/or any likewise device providing high precision positioning and/or translation along at least two and/or three perpendicular axes of motion. A target egg may be placed on its side on the surface and a waiting period of a few minutes may be afforded during which the germinal disc moves towards the upper part of the egg against gravitation due to its relative lower density compared to the yolk. Thus, an initial estimation of the whereabouts of the germinal disc may be made as being located at or near the top-most portion of the egg's side facing upwards. However, there may be a margin of error of up to about 1.0 centimeters from the actual location of the germinal disc. The reflectance and/or emission measurements may begin at a starting point where the irradiation beam and objective focus on the eggshell at locations spaced apart and with the supposed location of the germinal disc therebetween. The egg may be moved in the directions of its longitudinal axis and/or latitudinal axis parallelly to the surface in steps of between about 0.5 millimeters and about 1.0 millimeters along a line trajectory for a series of repeated measurements. The changes in the reflectance and/or emission may be analyzed to determine a location of the germinal disc and/or a size thereof.

In some embodiments, the measured intensities, optionally being normalized, may be plotted as a function of location on the shell of the egg. For example, the plot may be a profile of a respective scan line along one of the longitudinal and latitudinal axes of the egg. At locations incident on a line segment which intersects with the germinal disc, a peak in the measured intensities may be observed. The width of the peak may be proportional to and/or a function of the size of the germinal disc along the scan line. In a case where the scan line and the diameter of the germinal disc coincide, the width is maximal. In fertile eggs the maximal width is significantly larger than in non-fertile eggs. The width may be compared to a threshold value, such that if exceeded then the egg may be determined to be fertile. Otherwise, the width may be recorded to serve as a lower bound for an estimation of the diameter of the germinal disc, and a maximal width may be searched for over additional iterations in which several candidates may be detected and recorded in a similar manner. In a case of a circular germinal disc, a single additional scan along a perpendicular line through the peak's maximum point location may supposedly find the diameter. However, the germinal disc might not have a shape of perfect circle, and further scans may be required. Additionally or alternatively, scan-like iterations applying a plurality of different irradiance and/or collection angles may be performed and analyzed similarly in determination of the maximal width observed for peaks of variations in the measured intensities as a function of the angle of irradiance and/or collection.

The disclosed subject matter is advantageous over pre-existing tools and/or techniques in that it provides for determination of a fertility state of an egg and/or other conditions thereof in a non-invasive manner, such as by optical measurement according to some embodiments, thus preserving the egg intact and avoiding potential harm as may occur in some known invasive detection procedures involving removal of the egg's shell and/or the like.

The disclosed subject matter may be utilized to detect a state and/or condition of an egg at any stage and even as soon as it has just been laid, thus saving time and/or cost of its keep where unnecessary until such determination may be made.

Before explaining at least one embodiment in detail, it is to be understood that embodiments are not necessarily limited in its application to the details of construction and the arrangement of the components and/or methods set forth in the following description and/or illustrated in the drawings and/or the Examples. Implementations described herein are capable of other embodiments or of being practiced or carried out in various ways.

Embodiments may be a system, a method, and/or a computer program product. The computer program product may include a computer readable storage medium (or media) having computer readable program instructions thereon for causing a processor to carry out aspects of the embodiments.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of embodiments may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of embodiments.

Aspects of embodiments are described herein with reference to flowchart illustrations and/or block diagrams of methods, apparatus (systems), and computer program products according to embodiments. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

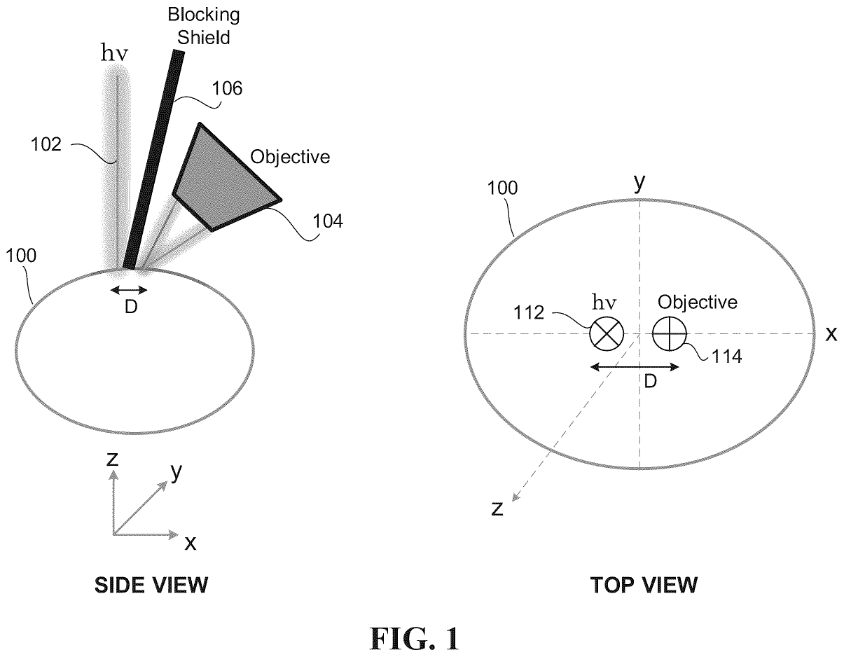

Reference is now made to FIG. 1 which is a schematic illustration of an exemplary measurement arrangement for electromagnetic detection of a state of an egg, according to some embodiments. For ease of understanding, FIG. 1 illustrates a side view and a top view of the egg and respective arrangement during measurement, in accordance with some embodiments of the disclosed subject matter.

As shown in FIG. 1, a target egg such as 100 may be placed on its size on a high precision 3-axis positioning surface such as an XYZ stage (not shown) and/or the like. An irradiating beam such as 102 may be emitted from an irradiation source (not shown), e.g., a light source providing an illumination beam and/or the like. The irradiation source may be and/or comprise a laser light source, and/or may optionally be and/or comprise a pulsated and/or time resolved irradiation functionality, such as for example, a picosecond laser and/or the like, capable of producing a plurality of short pulses of light and/or the like. Additionally or alternatively, the electromagnetic radiation emitted by the irradiation source may be continuous.

The irradiating beam 102 may be directed at and propagate towards a specified location on the shell of the egg 100, as may be designated for a current measurement, optionally as one of a plurality of iterations within one or more scans, each comprising a series of locations, angles, and/or the like. The irradiation beams impinging on the egg 100 may be focused on and/or confined to a predetermined region of the egg, to provide electromagnetic radiation that interacts with organelles in a medium within the egg 100, e.g., the germinal disc and its yolk matrix, for obtaining information such as size and/or morphology of the germinal disc indicative of a fertility state and/or health condition of the egg 100.

In some embodiments, the irradiation source may be and/or comprise a tuneable irradiation source, which may be controllable by a controller optionally coupled to a computer to provide light in a set of wavelengths and/or intensities that may be focused on the target egg 100 to interact with a region in an egg medium that provides desired inter-egg information (fertility and/or health). The controller may be chosen such that each impinging light beam 102 on the egg 100 comprises light at tuneable wavelengths for interactions between electromagnetic radiation and organelles in the germinal disc. Optionally the irradiating beam 102 may be provided at a wavelength of about 490 nanometres. More optionally, the irradiating beam 102 may be provided at a plurality of wavelengths comprising at least one wavelength of about 490 nanometres and at least one other wavelength of about 540 nanometres.

In some embodiments, the irradiating beam 102 may be adjusted to a respective one of a plurality of irradiation angles at each measurement. Alternatively, the irradiation angle may be fixed, and may be for example at about 0° such as illustrated on FIG. 1.

An objective such as 104 with a functionality of gathering and/or redirecting beams of electromagnetic radiation, which may be for example a lens, prism, and/or any likewise optical device, may be directed at another specified (and optionally different) location on the shell of the egg 100 and collect therefrom scattering, reflectance, emission, and/or the like of electromagnetic radiation, as may result from irradiation of the egg 100 by the irradiating beam 102.

In some embodiments, the objective 104 may be adjusted to a respective one of a plurality of collection angles at each measurement. Alternatively, the collection angle may be fixed, and may be for example at about 30° such as illustrated on FIG. 1.

As shown on FIG. 1, the irradiating beam 102 and objective 104 may be spaced apart by a predetermined distance D between a respective irradiation point 112 and a respective collection point 114 corresponding thereto. In some embodiments, the distance D may range between about 0.5 millimeters and about 5 millimeters, for example, the collection point 114 may be at another location on the shell removed from the irradiation point 112 by about 3 millimeters and/or the like. The irradiation and collection points 112 and 114 may be set initially according to an estimated location of the germinal disc of the egg 100, i.e., circa a top-most point on the shell of the egg 100 when placed on its side. For example, as shown on FIG. 1, the irradiation and collection points 112 and 114 may be located at two opposite sides around an intersection point of the longitudinal and latitudinal axes of the egg 100 on the top portion of the shell, such that the germinal disc may supposedly be in-between the two points 112 and 114. The egg 100 may be moved along the latitudinal axis, denoted as the y-direction on FIG. 1, and/or along the longitudinal axis, denoted on FIG. 1 as the x-direction, for subsequent measurements, optionally in a line trajectory, and more optionally with steps of between about 0.5 and about 1 millimeter.

In some embodiments, a blocking shield such as 106 may be placed between the irradiating beam 102 and objective 104, for example, between the irradiation point 112 and collection point 114, in order to prevent from reflectance from the shell of the egg 100 to reach at the collection point 114 and/or the objective 104. The blocking shield 106 may be made from one or materials with absorption capabilities of at least some of the electromagnetic radiation of the irradiance beam 102. Additionally or alternatively, selective capturing modes such as for example using polarization, filtering, coloring, and/or the like, optionally in combination with one or more irises and/or likewise mechanical and/or optical aids, may be employed so that saturation and collection of unwanted scattering may be avoided. The blocking shield 106 may be moved with the egg to a respective location in a subsequent measurement so as to maintain its relative position between the beam 102 and objective 104 and/or the irradiation and collection points 112 and 114.

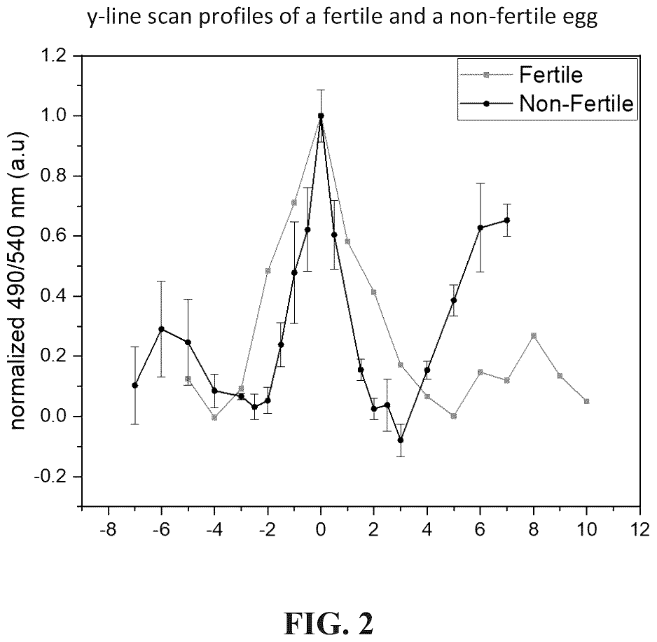

Reference is now made to FIG. 2 which is a schematic graph representing exemplary scan profiles at a latitudinal scan line of a fertile and a non-fertile egg, according to some embodiments.

FIG. 2 shows two scan profiles obtained for a fertile and a non-fertile egg at a scan line along a latitudinal axis of an egg, such as the latitudinal axis denoted as y-direction on FIG. 1 herein. The normalized scan values, i.e. rations of measured intensities at the wavelengths of 490 and 540 nanometers respectively, are plotted as a function of a location on the eggshell along the scan line (i.e., y-axis) relative to the origin, which may be set as the initial estimated location of the germinal disc as described herein. The position of 0 millimeters may supposedly be the location of the germinal disc on yolk in each of the eggs, as may be indicated by a peak in each of the profiles as depicted on FIG. 2. The width of the peak, which may optionally be determined using the full width at half maximum (FWHM) and/or a likewise measure may be utilized in estimation of the diameter of the germinal disc of the egg at hand, for example, as shown on FIG. 2, the FWHM of the non-fertile egg is about 1.66 millimeters, and the FWHM of the fertile egg is 3.44 millimeters, which roughly conform to average values of about 4 millimeters for a diameter of a fertile germinal disc and about 1 millimeter for a diameter of a non-fertile germinal disc.

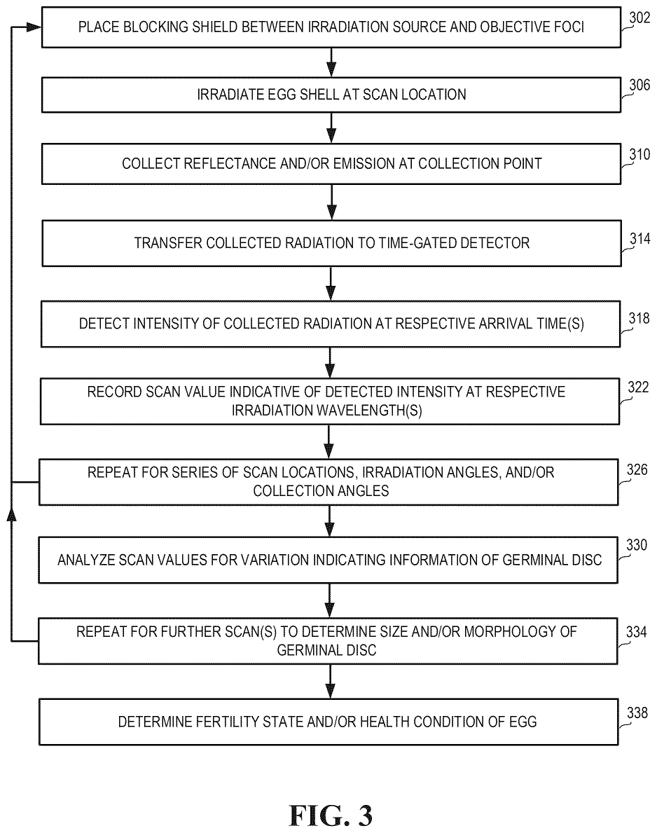

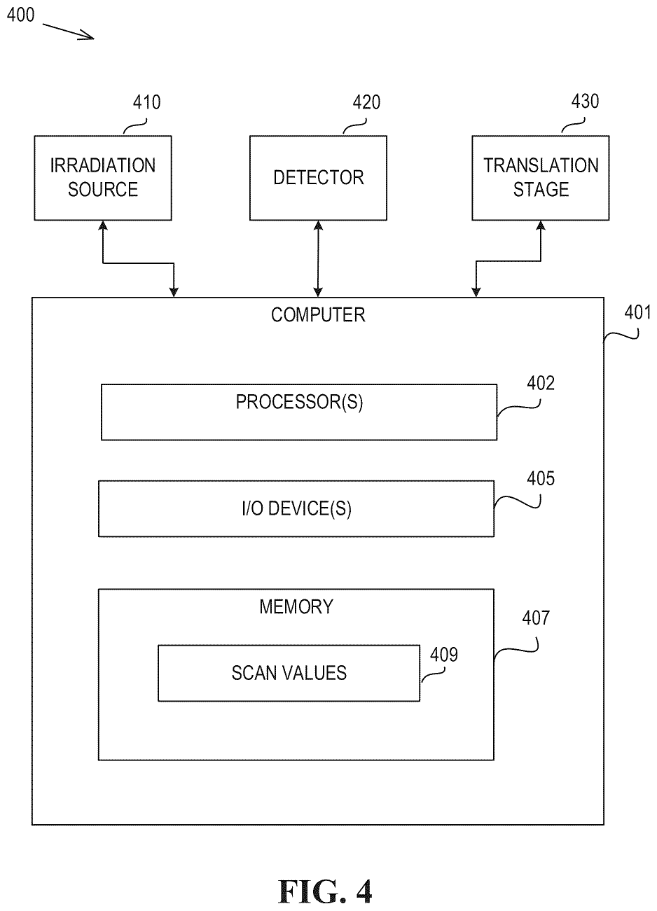

Reference is now made to FIG. 3 which is a flowchart schematically representing an optional flow of operations for electromagnetic detection of a state of an egg, according to some embodiments.

At 302, a blocking shield (such as 106 of FIG. 1) may be placed between respective points on a shell of an egg (such as irradiation and collection points 112 and 114 of FIG. 1) at which an irradiation source and an objective (such as 104 of FIG. 1) may be focused respectively.

At 306, the egg may be irradiated at a scan location on the shell where an irradiating beam (such as 102 of FIG. 1) may be focused.

At 310, reflectance and/or emission emanating from the egg may be collected at a collection point where the objective may be focused.

At 314, the electromagnetic radiation (i.e., reflectance and/or emission) collected at the collection point at 310 may be transferred to a time-gated detector.

At 318, an intensity of the electromagnetic radiation collected at 310 may be detected at one or more respective arrival times to the time-gated detector relative to a time of the irradiating at 306. In some embodiments, the intensity may be measured for each of one or more wavelengths used for the irradiating at 306.

At 322, a respective scan value indicative of the intensity measured for each wavelength at 318 may be recorded. In some embodiments, the recorded scan value may be a normalized scan value which may be calculated, e.g., as a ratio of measured intensities at two distinct wavelengths and/or the like.

At 326, one or more of the acts carried out through 302 to 322 may be repeated for each of a series of scan locations, irradiation angles, collection angles, and/or the like.

At 330, the scan values as recorded at 322 respective of plurality of locations, angles, and/or the like may be analyzed for variation indicating information of the egg's germinal disc. In some embodiments, the scan values may be plotted as a function of location, angle, and/or the like and a maximizing argument and/or a full width at half maximum (FWHM) value and/or any likewise measure may be determined. The FWHM may be used as an estimator of a diameter of the germinal disc, according to some embodiments.

At 334, one or more of the acts carried out through 302 to 330 may be repeated for one or more further scans as may be required in order to determine the size (i.e., diameter) and/or additional morphological information of the germinal disc, e.g., by analysis of other and/or additional scan values thus obtained, which may be performed similarly as in 330. Otherwise, the procedure may skip to 338.

At 338, a fertility state and/or health condition of the egg may be determined according to the information of the germinal disc as determined by the analysis of scan values at 330 and/or 334.

Reference is now made to FIG. 4 which is a schematic block diagram of an exemplary system for electromagnetic detection of a state of an egg, according to some embodiments.

A system 400 for electromagnetic detection of a state of an egg may comprise a computer such as 401, which may be implemented as, for example, a standalone unit, a server, a computing cloud, a desktop computer, a laptop computer, a tablet computer, a smart phone, a wearable computer, a mainframe computer, a quantum computer, and/or the like. The computer 401 may be implemented as a customized unit that includes locally stored software and/or hardware that perform one or more of the acts described with reference to FIG. 3 herein. Alternatively or additionally, the computer 401 may be implemented as code instructions loaded on an existing computing device. Alternatively or additionally, the computer 401 may be implemented as hardware and/or code instructions (e.g., an accelerator card) installed and/or integrated within an existing computing device.

The computer 401 may comprise one or more processors such as 402, which may be implemented as, for example, a central processing unit(s) (CPU), a graphics processing unit(s) (GPU), field programmable gate array(s) (FPGA), digital signal processor(s) (DSP), and application specific integrated circuit(s) (ASIC). Processor(s) 402 may include one or more processors (homogenous or heterogeneous), which may be arranged for parallel processing, as clusters and/or as one or more multi core processing units.

In some embodiments, the computer 401 may comprise a network interface (not shown) for transmission and/or receipt of data over a network (not shown) and/or other suitable communication channel. The network may be any type of data network, for example, a local area network (LAN), a wireless LAN, a wide area network (WAN), or the connection may be made to an external computer, for example through the Internet using an Internet Service Provider (ISP) and/or any other type of computer network. The wireless LAN may use one or more wireless protocols, including Bluetooth, Bluetooth low energy (BLE), 802.11 compliant wireless local area network (WLAN), and/or any other wireless LAN protocol. The network may use networking protocols, for example Transmission Control Protocol and Internet Protocol (TCP/IP), Asynchronous Transfer Mode (ATM), asymmetric digital subscriber line (ADSL), and/or any other networking protocol. The network may comprise one or more routers, wireless routers, hubs, smart hubs, switches, smart switches, and/or any other type of networking equipment.

The computer 401 may comprise one or more input and/or output (I/O) devices such as 405 for receiving input from and/or providing output to a user. Exemplary I/O device(s) 405 of computer 401 may comprise one or more of: a touchscreen, a display, a keyboard, a mouse, voice activated software using speakers and microphone, a printer, a touchpad, game controllers, haptic devices, and/or the like. Additionally or alternatively, one or more standalone devices communicating with processor(s) 402, e.g., via the network, may serve as I/O device(s) 405, for example, a mobile and/or stationary computing device such as a smart phone, a tablet computer, a laptop computer, a desktop computer, a wearable computer, and/or the like, running a suitable application program, may establish communication (e.g., cellular, network, short range wireless) with the processor(s) 402 using a communication interface (e.g., network interface, cellular interface, short range wireless network interface). The user may input data and/or receive data outputted by the respective device, e.g., by entering and/or viewing data on a display of the computing device (e.g., a smart phone), optionally via a graphical user interface (GUI) application and/or the like.

The computer 401 may comprise and/or be coupled to a memory and/or data storage device such as 407, which may be configured to store code instructions executable by processor(s) 402, for example, a random access memory (RAM), a read-only memory (ROM), and/or a storage device, for example, a non-volatile memory, magnetic media, semiconductor memory devices, a hard drive, a removable storage (e.g., a flash drive), optical media (e.g., DVD, CD-ROM), a virtual drive and/or storage service which may be implemented as, for example, a remote server and/or computing cloud (e.g., accessed via a network connection), and/or the like. Memory 407 may store code instructions that implement one or more of the acts described with reference to FIG. 3 herein. Alternatively or additionally, one or more of the acts described with reference to FIG. 3 herein may be implemented in hardware.

The system 400 may further comprise an irradiation source such as 410, a detector such as 420, and a translation stage such as 430, all of which may be coupled to and controllable by the computer 401 to perform one or more of the acts described with reference to FIG. 3 herein.

The irradiation source 410 may be operated by the computer 401 to emit a beam of electromagnetic radiation directed towards and preferably focused at a specified location on a shell of an egg placed on the translation stage 430. The computer 401 may control and/or adjust one or more further parameters of the irradiation source 410 such as for example, an irradiation angle, an irradiation wavelength, and/or the like. In some embodiments, the irradiation source 410 may be adapted for irradiating the egg by a plurality of pulses, which one or more parameters thereof such as, for example, a number of pulses, a width of each pulse, and/or the like, may similarly be controlled and/or adjusted by the computer 401.

The detector 420 may be operated by the computer 401 to capture and measure intensity of electromagnetic radiation emanating from the egg at a collection point which may correspond to the irradiance location.

The detector 420 may comprise and/or be coupled to at least one of the components: collecting optics (such as for example the objective 104 of FIG. 1 herein), a streak camera, a trigger, a delay unit, a power supply, and a controller, optionally controlled by the computer 401. Electromagnetic radiation outputted from the germinal disc and optionally its surroundings in an egg irradiated by irradiation source 410, e.g., reflected light (and/or absorbed light, and/or transmitted light and/or scattered light), may be collected via the collecting optics and transferred to an input of the streak camera of the detector 420, preferably a time gated detector such as a picosecond laser detector and/or the like, configured to capture reflectance and/or emission from the germinal disk over a time window set by the streak camera. The intensity of the reflected light (and/or absorbed light, and/or transmitted light) may depend on the inspection (i.e., collection) angle.

The collecting optics of the detector 420 may comprise a lens system for focusing the outputted light, and/or optical fibers. The detector 420 may be operated by the computer 401 to focus on the collection point which may be removed from the irradiance point by a predetermined distance, optionally by controlling and/or manipulating the collecting optics and/or lens system therein. Optionally the computer 401 may control and/or adjust a collection angle at which the electromagnetic radiation at the collection point may be captured by the detector 420. The size of the time window (i.e., duration) may determine the time interval selected and displayed at the detector.

In order to design the streak camera of the detector 420 to perform the capturing of the light from the germinal disc in the time window as discussed herein, a delay unit may be provided. The delay unit may comprise an optical setup through which light may be transferred such that light may arrive at an input of the detector 420 in a delay corresponding to the specified time window, e.g., through a mirror and/or a set of mirrors or through an optical fiber, such that the light may arrive at the streak camera of the detector 420 at a delay. The delay may be determined by the additional path or by the fiber's length, such that light reflected from the egg's shell may fall out of the streak camera's measurement window and light originating from the germinal disc may fall inside the streak camera's measurement window.

In order to control the time delay for generating operation of measurement by the streak camera, a signal (e.g., pulse of light) from the irradiation source 410 may be transmitted to the trigger of the detector 420, which may function to trigger a signal to the delay unit of the detector 420 at a time window corresponding to light arriving from the germinal disc. The time window may be programmable and controlled by the computer 401 and/or electronic components, which may be included within the irradiation source 410. The trigger signal may be fed into the streak camera of the detector 420.

In some embodiments, the irradiation source 410 may comprise an electronic triggering.

Alternatively, no delay unit may be included in the system 400 according to the disclosed subject matter. The delay unit of the detector 420 may be implemented and/or utilized according to the disclosed subject matter, by using a suitable geometry, in which a distance between the relevant components of the system produces an optical delay.

The streak camera of the detector 420 may utilize a streak tube. The streak camera may comprise a photocathode for producing emission of electrons in proportion to intensity of the incident light. The streak camera may be utilized for forming a streak image of light received at a desired time window corresponding to the time during which the streak image is formed. A streak camera image may be collected for each irradiation wavelength, each image having two dimensions of a time and a distance along a line corresponding to different locations. In addition, the irradiated beam power at each wavelength may also be recorded and used as reference for corrections (e.g., normalization) and/or the like.

At the computer 401, the measured intensity of the reflectance and/or emission from the germinal disc at each of one or more irradiation wavelengths used may be received from the detector 420 and may be stored in the memory 407 as respective scan value(s) 409 for the respective irradiance location, irradiance angle and/or collection angle at hand. Additionally or alternatively, the processor(s) 402 may calculate a respective normalized scan value, for example, as a ratio of measured intensities at different irradiation wavelengths, and record all the (normalized) scan values 409 in the memory 407.

The processor(s) 402 may perform analysis of the scan values 409 stored in the memory 407 and determine a state of the egg according to a determination of at least one of a size and morphology of the germinal disc as disclosed herein. Additionally or alternatively, processor(s) 402 may determine a location of the germinal disc and operate the irradiation source 410, detector 420, and/or translation stage 430 for performing further measurements and/or detailed scan(s) and obtaining other and/or further scan values for further analysis and/or processing as may be required in order to determine the state of the egg.

In some embodiments, the system 400 may comprise a mount (not shown) for placing a blocking shield (such as 106 of FIG. 1) to prevent at least in part from reflectance from the shell of the egg to reach the collection point and/or the detector 420. The mount may be coupled to an actuator (not shown) such as a robotic arm and/or the like, which may be controllable by the computer 401 for displacing the blocking shield to a respective position in a beginning of a measurement at a subsequent location on the shell of the egg. Additionally or alternatively, the irradiation source 410 and/or collecting optics of the detector 420 may be configured for selectively capturing electromagnetic radiation emanating from the egg at the collection point, by using one or blocking and/or restriction measures, such as for example, polarization, colorization, filtering, and/or the like, in order to eliminate and/or reduce the shell reflectance from interfering with detected signals.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant electromagnetic irradiation and/or time gated detection tools and/or techniques will be developed and the scope of the terms electromagnetic irradiation and/or time gated detection is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment may include a plurality of “optional” features unless such features conflict.

Throughout this application, various embodiments may be presented in a range format. It should be understood that the description in range format is merely for convenience and brevity and should not be construed as an inflexible limitation on the scope of embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of embodiments, which are, for clarity, described in the context of separate embodiments, may also be provided in combination in a single embodiment. Conversely, various features of embodiments, which are, for brevity, described in the context of a single embodiment, may also be provided separately or in any suitable subcombination or as suitable in any other described embodiment. Certain features described in the context of various embodiments are not to be considered essential features of those embodiments, unless the embodiment is inoperative without those elements.

Although embodiments have been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for detecting a state of an egg, comprising:
obtaining a plurality of scan values by performing, over a plurality of iterations respective of at least one of a plurality of scan locations on a shell of an egg, a plurality of irradiation angles, and a plurality of collection angles:
irradiating the egg with electromagnetic radiation at a respective location of the plurality of scan locations and a respective one of the plurality of irradiation angles;
capturing and measuring intensity of electromagnetic radiation emanating from the egg at a collection point corresponding to the respective location of irradiance and a respective one of the plurality of collection angles, using a time-gated detector; and
calculating and storing a respective scan value for the respective location and angles indicative of the intensity measured for at least a portion of a plurality of arrival times of the captured electromagnetic radiation to the time-gated detector relative to a time of irradiation;
analyzing the plurality of scan values for at least the portion of the plurality of arrival times to determine information of a germinal disc of the egg; and
determining a state of the egg according to the information.

2. The method of claim 1, wherein for at least one portion of the plurality of scan locations, a respective portion of the plurality of scan values are analyzed to determine a local extremum indicative of incidence on a line intersecting with the germinal disc, wherein at least another portion of the plurality of scan locations are determined in accordance with a respective one of the at least one portion of the plurality of scan locations at which the local extremum is obtained.

3. The method of claim 1, wherein the plurality of scan locations on the shell of the egg being positioned along one of a longitudinal axis and a latitudinal axis of the egg and spaced apart from one another by a distance ranging between about 0.5 millimeter and about 1 millimeter.

4. The method of claim 1, wherein the irradiating comprising applying a plurality of electromagnetic pulses having a width of between about 100 femtoseconds and about 100 picoseconds.

5. The method of claim 1, wherein the irradiating comprising applying at least one wavelength at which a difference between at least one of a reflectance and emission of electromagnetic radiation from the germinal disc, and at least one of the reflectance and emission from a matrix of the germinal disc within the egg, is discernable.

6. The method of claim 5, wherein the irradiating comprising applying a plurality of wavelengths, wherein for at least a portion of the plurality of wavelengths the difference is by a first magnitude, wherein for at least an additional portion of the plurality of wavelengths the difference is by a second magnitude distinct from the first magnitude.

7. The method of claim 6, wherein the portion comprising a wavelength of about 490 nanometers, wherein the additional portion comprising a wavelength of about 540 nanometers.

8. The method of claim 6, further comprising normalizing captured intensities for the portion using respective captured intensities for the additional portion.

9. The method of claim 1, wherein the collection point being removed from the respective location of irradiance by a distance of between about 0.5 millimeters and about 5 millimeters.

10. The method of claim 1, wherein the irradiating and capturing further comprising preventing at least in part from reflectance of electromagnetic radiation from the shell of the egg to reach the collection point.

11. The method of claim 1, wherein analyzing the plurality of scan values and determining the state of the egg comprising:
determining a peak and a width thereof along at least one line trajectory on the shell of the egg; and
determining a fertility state of the egg according to at least one of:
determining a diameter of the germinal disc of the egg according to a maximal width determined from the at least one line trajectory, and
determining a lower bound of the diameter in response to the width along a respective one of the at least one line trajectory exceeding a threshold.

12. The method of claim 1, wherein obtaining the plurality of scan values is performed at a spatial resolution for which a morphology of the germinal disc is discernable, wherein analyzing the plurality of scan values comprising detecting at least one morphological characteristic of the germinal disc, wherein determining the state of the egg comprising determining a health condition of the egg.

13. The method of claim 1, wherein the egg is a freshly laid egg for which a time elapsed from laying is less than an hour.

14. The method of claim 1, wherein the plurality of iterations being performed respective of at least one of the plurality of scan locations and the plurality of collection angles, and wherein the irradiating being at an irradiation angle of about 0°.

15. The method of claim 1, wherein the plurality of iterations being performed respective of at least one of the plurality of scan locations and the plurality of irradiation angles, and wherein the capturing being at a collection angle of about 30°.

16. A system for detecting a state of an egg, comprising:
an irradiation source adapted for irradiating an egg with electromagnetic radiation at a respective location of a plurality of scan locations on a shell of an egg and a respective one of the plurality of irradiation angles;
a time-gated detector adapted for capturing and measuring intensity of electromagnetic radiation emanating from the egg at a collection point corresponding to the respective location of irradiance and a respective one of the plurality of collection angles;
a processing circuitry coupled to a memory and adapted to execute a code for:
obtaining a plurality of scan values by operating the irradiation source and time-gated detector over a plurality of iterations respective of at least one of the plurality of scan locations, the plurality of irradiation angles, and the plurality of collection angles;
analyzing the plurality of scan values for at least the portion of the plurality of arrival times to determine information of a germinal disc of the egg; and
determining a state of the egg according to the information.

17. The system of claim 16, further comprising a blocking shield for preventing from reflectance of electromagnetic radiation from the shell of the egg to reach the collection point.

18. The system of claim 16, further comprising an objective lens for collecting and transferring the electromagnetic radiation emanating from the egg at the collection point to the time-gated detector.

19. The system of claim 16, further comprising a translation stage for positioning the egg relative to the irradiation source for the irradiating at the plurality of scan locations.
